(19) United States
(12) Patent Application Publication
(10) Pub. No.: US 2012/0323069 A1
(43) Pub. Date: Dec. 20, 2012

(54) ENDOSCOPE SYSTEM ADAPTER

(76) Inventor: Christopher A. Stout, San Bruno, CA (US)

(21) Appl. No.: 13/163,617

(22) Filed: Jun. 17, 2011

(51) Int. Cl. A61B 1/018

(52) U.S. Cl. 600/104

(57) ABSTRACT

An adaptor and adaptor kit are described in which the adaptors may be configured to be removably coupled to a working channel of an endoscope system, such as a hysteroscope system. The adaptors may include an elongate body having a central lumen extending between a distal end and a proximal end of the elongate body, and a bend along a length of the elongate body between the distal and proximal ends. The adaptors may be configured to accommodate variations in patient anatomy, such as variations in the size and shape of a uterus, and location of the ostia to the fallopian tubes. A plurality of non-identical adaptors having a number of different characteristics may be sold in a kit so that particular adaptor can be selected for use with a particular patient anatomy.

### BACKGROUND

Embodiments of the present invention relate to an endoscope system and methods. More specifically, embodiments of the present invention relate to a hysteroscope system and adaptor for transcervically accessing fallopian tubes.

Female contraception and sterilization may be effected by hysteroscopic sterilization in which an insert is transervically introduced into a fallopian tube to inhibit conception. Devices, systems and methods for such an approach have been described in various patents and patent applications assigned to the present assignee. For example, U.S. Pat. No. 6,526,979, U.S. Pat. No. 6,634,361, U.S. Patent application Ser. No. 11/165,733 published as U.S. Publication No. 2006/0293560 and U.S. patent application Ser. No. 12/605,304 describe transcervically inserting an insert (also referred to as implant and device) into an ostium of a fallopian tube and mechanically anchoring the insert within the fallopian tube. One example of such an assembly is known as “Essure”® from Conceptus, Inc. of Mountain View, Calif. Tissue in-growth into the “Essure”® insert provides long-term contraception and/or permanent sterilization without the need for surgical procedures.

An important aspect of hysteroscopic sterilization requires navigating through the uterus to locate the tubal ostia. A hysteroscope is a long, narrow telescope connected to a light source to illuminate the area to be visualized. In order to gain access to the uterus a speculum is generally inserted into the vagina and an elongated sheath of a hysteroscope system is then inserted through the vagina and cervical canal and, under direct visualization, advanced into the uterine cavity. A gas or liquid is often injected through an inflow port in the hysteroscope system and into the uterus to distend the uterine cavity, enabling visualization and operation in an enlarged area. Due to patient sensitivity in the cervical canal, it is preferable to keep the elongated sheath of the hysteroscope system at the 6 and 12 o'clock positions and avoid side-to-side motions against the cervical canal. Once the tubal ostia are located a delivery catheter may then be advanced through a working channel of the hysteroscope system and into a fallopian tube to deliver the insert. In principle, there are two different types of hysteroscope systems available: rigid and flexible.

Rigid hysteroscope systems generally incorporate glass optics and achieve the best available resolution. A rigid hysteroscope system is illustrated in FIGS. 1A-1C. As illustrated, a rigid hysteroscope system 100 may include an eyepiece 102, an access port 104, an inflow port 106, an outflow port 108, and rigid elongated sheath 110. Referring to FIGS. 1B-1C, glass optics 112, a working channel 114, an inflow channel 116 and outflow channel 118 may all extend through and terminate at a distal end of the rigid elongated sheath 110. Side outflow openings 120 may be included along a distal end region of the rigid elongated sheath 110. Because the optics are glass, the glass optics 112 and elongated sheath 110 are necessarily rigid and the telescope angle of the glass optics 112 is fixed. During insertion through the cervical canal and into the uterine cavity small up and down and side-to-side motions against the cervical canal are often necessary, resulting in patient discomfort.

Flexible hysteroscope systems typically include fiber optics and a distal tip which is deflectable over a range of 120-160 degrees. The flexible contour of flexible hysteroscopes may allow for navigation through an irregularly shaped uterus with reduced side-to-side motions against the cervical canal thereby avoiding the discomfort which can be associated with inserting a rigid hysteroscope system through the cervical canal and navigating it within a uterine cavity. The flexibility is generally enabled by the use of fiber optic bundles which are more expensive than glass optics and do not achieve the same resolution as the glass optics of the rigid hysteroscope systems.

### SUMMARY

Embodiments of the present invention generally relate to an endoscope system and methods. More specifically, embodiments of the present invention generally provide a hysteroscope system and adaptor for transcervically accessing fallopian tubes.

In one embodiment of the present invention, an adaptor includes an elongate body, which may be a tubular elongate body, with a central lumen extending between a distal end and a proximal end of the elongate body and a bend along a length of the elongate body between the distal and proximal ends. The proximal end of the elongate body is configured to be removably coupled to a working channel of an endoscope system, which may be a rigid hysteroscope system. The bend may deflect the central lumen between 0 and 90 degrees. The bend may also be defined by a radius of curvature, for example between 0.020 inches to 2 inches. The elongate body may further include a straight portion, for example between 0 cm and 20 cm. The proximal end of the elongate body may be configured to be press fit into the working channel of an endoscope system. This may be accomplished by a taper on the proximal end. The adaptor may also or alternatively include a sleeve attached to the elongate body which is configured to slip over the rigid elongated sheath of the endoscope system. In one embodiment, the sleeve can include an elastic material such as silicone or urethane on the inside of the sleeve such that the elastic material springs onto the rigid elongated sheath of a rigid hysteroscope system. The elastic material may be an O-ring. In one embodiment, the sleeve can include protrusions along the length of the sleeve that are configured to align with the side outflow openings along the length of the rigid elongated sheath.

The elongate body may be formed of a variety of different materials. In accordance with some embodiments, the elongate body may be formed of a transparent material, or include a window of a transparent material. The transparent material may allow for improved visualization with glass optics of the endoscope system and manipulation of a delivery system through the central lumen of the adaptor. It is not required for the elongate body to include a transparent material, and the elongate body may be formed of an opaque material in accordance with other embodiments.

The elongate body may be formed of both rigid and flexible materials. For example, a rigid transparent material polymer such as polycarbonate, or a rigid transparent glass such as a tempered soda-lime glass, also known as pyrex® may be used. The elongate body may also be formed of a rigid opaque material such as stainless steel.

In an embodiment, the elongate body is formed of a flexible material. A broad range of flexibilities are possible. In an embodiment, the elongate body is formed of a polymer which is flexible to allow for easy cannulation of the cervical canal and potentially cause less discomfort to a patient when advancing through the cervical canal, yet retains the intended shape and bend when advanced into the uterus. In an embodiment, the elongate body is formed of thin walled steel which can be manually deformed to achieve a desired bend by the operator. In another embodiment, the elongate body may be formed of a shape memory polymer. In such an embodiment, the elongate body may be inserted through the cervical canal in a straight configuration thereby causing a minimum amount of discomfort to the patient during initial advancement, and reconfigure to a desired bend when inserted into a uterine cavity and warmed, for example by a distention fluid.

In an embodiment, the elongate body includes at least one adjustable joint which may be adjusted by the operator in order to obtain a desired bend. The adjustable joint may include a set screw to tighten the joint at a desired angle. The elongate body with an adjustable joint may include an inner sleeve defining the central lumen, and proximal and distal arms connected at the adjustable joint. In another embodiment, the elongate body includes a plurality of adjustable cylinders which can be adjusted by twisting by the operator in order to obtain a desired bend. The elongate body including a plurality of adjustable cylinders may include an inner sleeve defining the central lumen.

In an embodiment, a plurality of non-identical adaptors having a number of different characteristics may be assembled in a kit. Differentiation among the adaptors may be to fit working channels of various endoscope manufacturers or to obtain a suitable configuration for a particular patient anatomy. For example, the plurality of non-identical adaptors may include a plurality of different radii of curvature, a plurality of different angles of deflection, or a plurality of different straight portion lengths for the plurality of elongate bodies. A kit may include one or more straight adaptor extension lengths. A kit may include a plurality of non-identical adaptors having a number of combinations of different characteristics.

### BRIEF DESCRIPTION OF THE DRAWINGS

FIG. 1A illustrates an isometric view of a hysteroscope system.

FIG. 1B illustrates a close-up isometric view of a distal end of a hysteroscope system.

FIG. 1C illustrates a cross-sectional isometric view of a distal end of a hysteroscope system.

FIG. 2A illustrates a side view of an adaptor in accordance with embodiments of the invention.

FIG. 2B illustrates an isometric view of an adaptor in accordance with embodiments of the invention.

FIG. 3 illustrates an adaptor attached to a hysteroscope system positioned within a uterus in accordance with embodiments of the invention.

FIG. 4A illustrates a top view of a hysteroscope system and adaptor at a 315 degree angle of orientation in accordance with embodiments of the invention.

FIG. 4B illustrates a top view of a hysteroscope system and adaptor rotated to a 135 degree angle of orientation in accordance with embodiments of the invention.

FIG. 4C illustrates a top view of a hysteroscope system and adaptor rotated to a 180 degree angle of orientation in accordance with embodiments of the invention.

FIGS. 5A-5B illustrate side views of adaptors with different angles of deflection in accordance with embodiments of the invention.

FIGS. 6A-6B illustrate side views of adaptors with different radii of curvature in accordance with embodiments of the invention.

FIGS. 7A-7B illustrate side views of adaptors with different straight section lengths in accordance with embodiments of the invention.

FIG. 8A illustrates a side view of an adaptor window in accordance with embodiments of the invention.

FIG. 8B illustrates a top view of an adaptor window in accordance with embodiments of the invention.

FIG. 9A illustrates an isometric view of an adaptor with an adjustable joint in accordance with embodiments of the invention.

FIG. 9B illustrates a top view of an adaptor with an adjustable joint in accordance with embodiments of the invention.

FIG. 10A illustrates a schematic view of an adjustable cylinder in accordance with embodiments of the invention.

FIGS. 10B-10C illustrate isometric views of an adaptor with a plurality of adjustable cylinders in accordance with embodiments of the invention.

FIG. 11A illustrates an isometric view of an adaptor fit into a working channel of a hysteroscope system in accordance with embodiments of the invention.

FIG. 11B illustrates a side view of an adaptor with a tapered proximal end in accordance with embodiments of the invention.

FIG. 11C illustrates an isometric view of an adaptor with a tapered proximal end in accordance with embodiments of the invention.

FIG. 11D illustrates an isometric view of an adaptor with a tapered proximal end and O-ring in accordance with embodiments of the invention.

FIG. 12A illustrates an isometric view of an adaptor sleeve fit over an elongated sheath of a hysteroscope system in accordance with embodiments of the invention.

FIG. 12B illustrates an isometric view of an adaptor sleeve with O-ring in accordance with embodiments of the invention.

FIG. 12C illustrates an isometric view of an adaptor sleeve with protrusions in accordance with embodiments of the invention.

FIG. 13 illustrates a schematic view of an adaptor kit in accordance with embodiments of the invention.

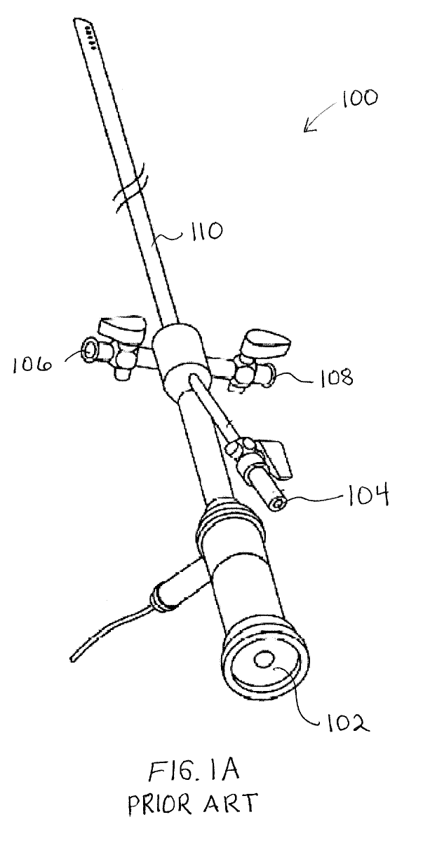

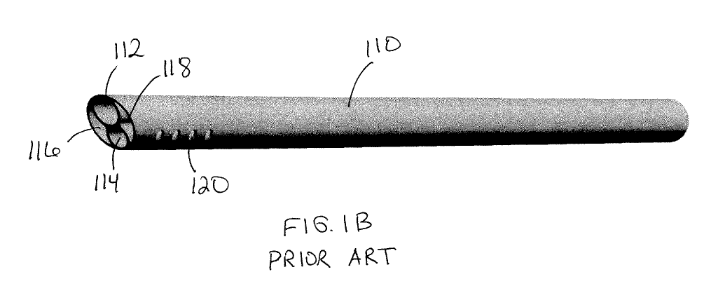

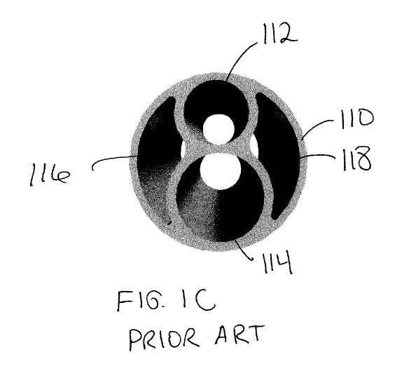

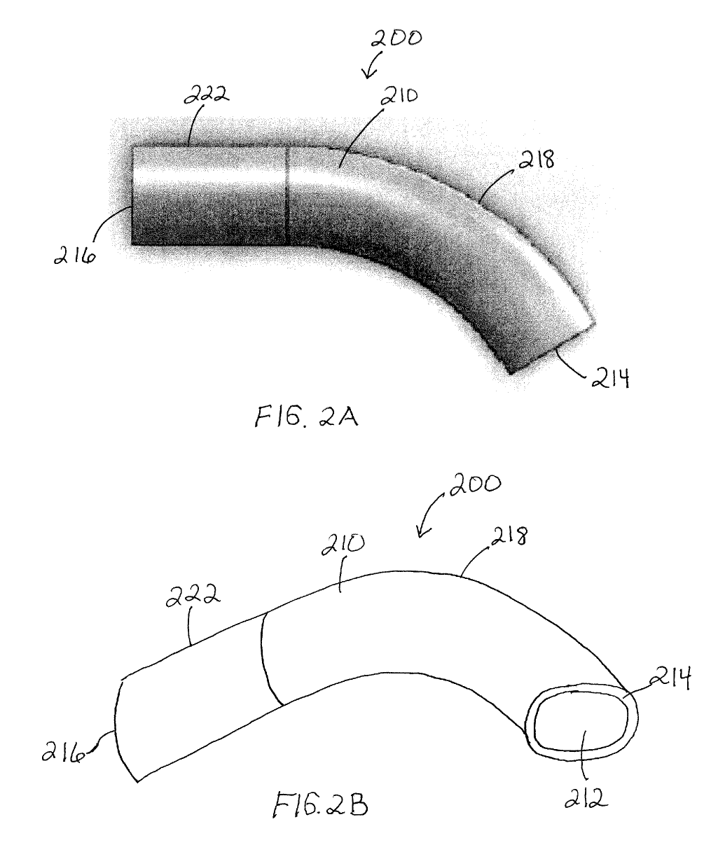

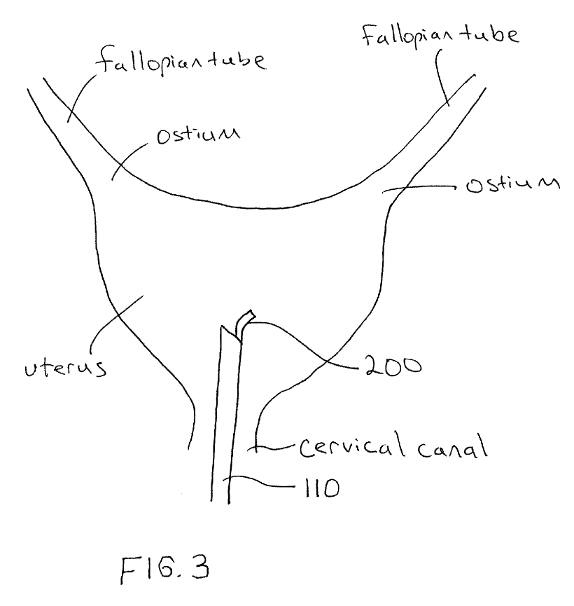

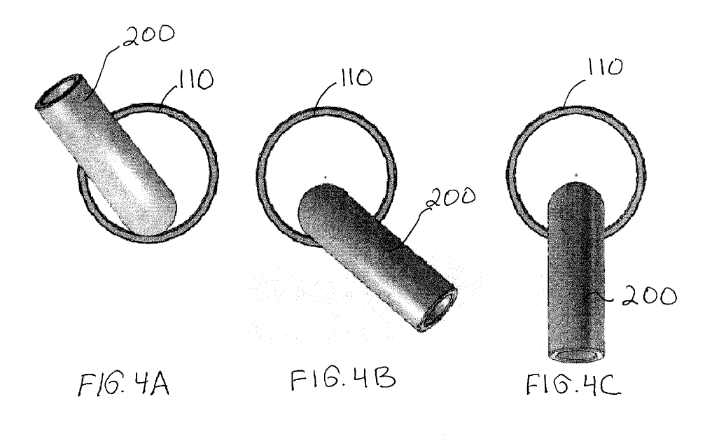

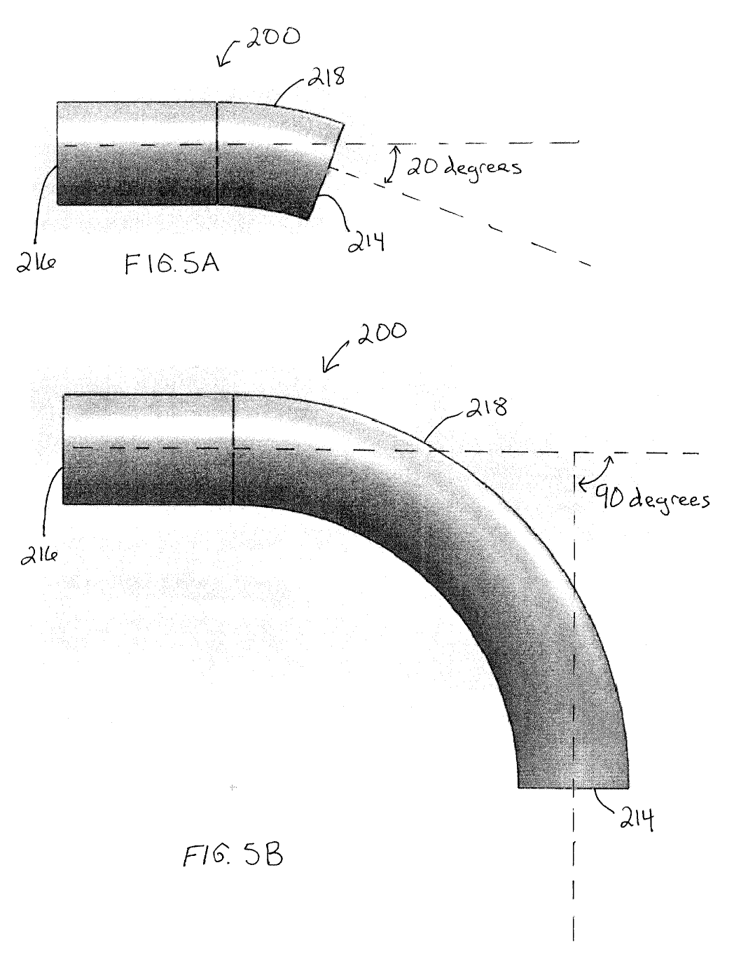

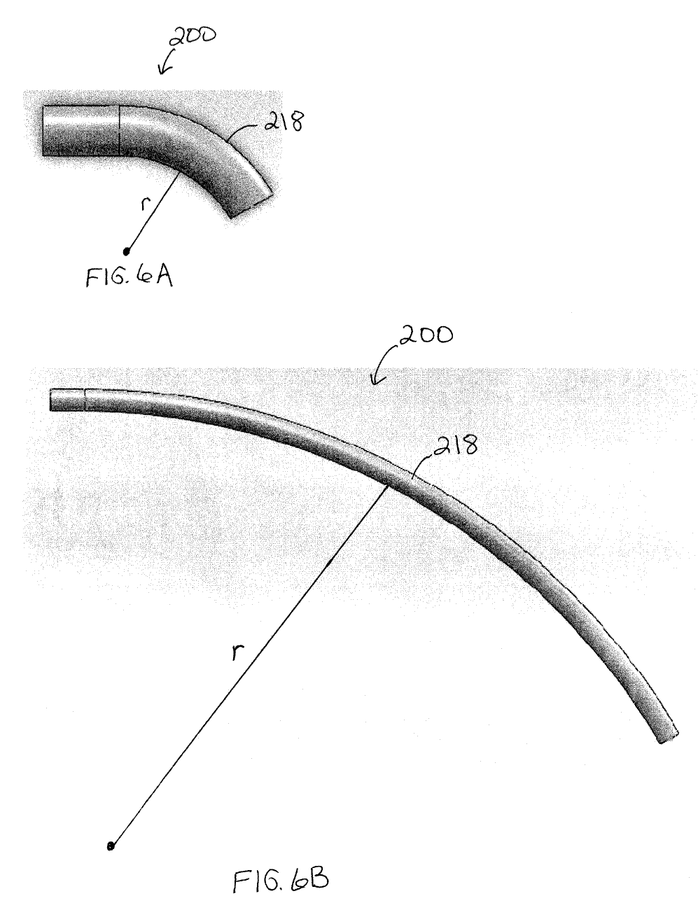

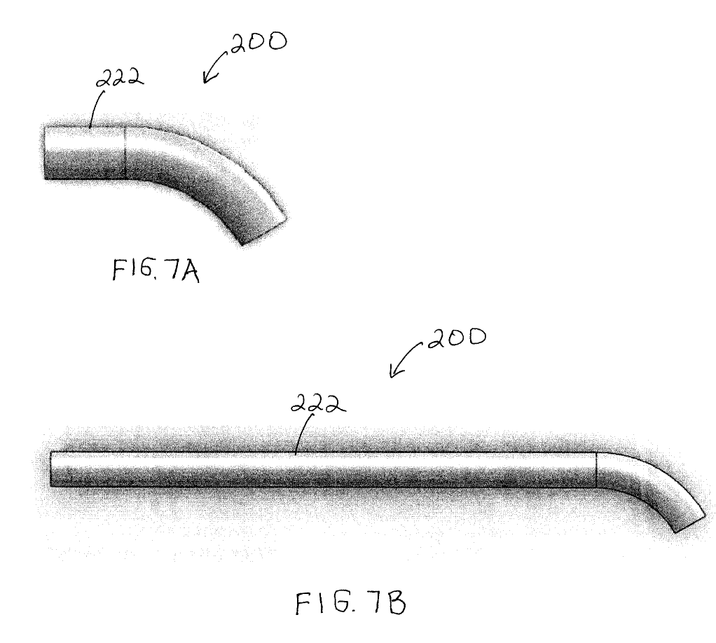

### DETAILED DESCRIPTION

Embodiments of the present invention generally relate to an endoscope system and methods. More specifically, embodiments of the present invention generally provide a hysteroscope system and adaptor for transcervically accessing fallopian tubes.

Various embodiments and aspects will be described with reference to details discussed below and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present invention.

In accordance with embodiments of the present invention, an adaptor is described which may be configured to attach to a working channel of a hysteroscope system, and allow a variety of catheters and devices to travel through it similarly as the working channel without significant resistance. While embodiments of the invention are described in detail with regard to a hysteroscope system, embodiments are not limited to such and may be compatible with suitable endoscope systems as well as other suitable optical surgical devices for accessing a reproductive system. Referring now to FIGS. 2A-2B, in accordance with embodiments of the invention, the adaptor 200 may include an elongate body 210 having a central lumen 212 extending between a distal end 214 and a proximal end 216 of the elongate body, and a bend 218 along a length of the elongate body between the distal and proximal ends 214, 216. In accordance with some embodiments the adaptor 200 may include a straight portion 222 along a length of the elongate body 210 between the distal and proximal ends 214, 216. The bend 218 and straight portion 222, if present, may be configured to accommodate variations among patients' anatomies, such as variations in the size and shape of the uterus and location of the ostia to the fallopian tubes. While embodiments of the invention describe a central lumen 212 extending between distal and proximal ends of an elongate body 210, it is understood that elongate body 210 may be a single component or include multiple components and materials. In accordance with embodiments of the invention, a catheter or other device may be advanced through the working channel of a hysteroscope system and through the central lumen 212 to access an area which may not have been as easily accessed without the adaptor 200. In accordance with many embodiments of the invention, elongate body 210 is a tubular elongate body, and the central lumen 212 extending therein may function as an extension of a tubular working channel of a hysteroscope system.

FIG. 3 is an exemplary illustration of an adaptor 200 attached to a hysteroscope system positioned within a uterus in accordance with embodiments of the invention. As illustrated, a rigid elongated sheath 110 of a hysteroscope system is positioned within a uterus and cervical canal at the 6 and 12 o'clock positions. The proximal end of the adaptor 200 is removably coupled with a working channel of the hysteroscope. The adaptor 200 further includes a bend which deflects the angle of orientation of the working channel of the hysteroscope system toward the ostium of a fallopian tube.

Referring now to FIGS. 4A-4C, the adaptor 200 can be rotated within the uterus to change the angle of orientation. For example, once the hysteroscope system and adaptor are positioned to align a central axis of the central lumen of the adaptor exiting the distal end of the adaptor with the ostium of a first fallopian tube, the hysteroscope system (including elongated sheath 110) can be rotated approximately 180 degrees to align the central lumen of the adaptor exiting the distal end of the adaptor 200 with the corresponding ostium of a second fallopian tube. FIG. 4A illustrates a top view of a hysteroscope system with an adaptor 200 at a 315 degree angle of orientation. FIG. 4B illustrates a 135 degree angle of orientation, and FIG. 4C illustrates a top a 180 degree angle of orientation accomplished by rotating the hysteroscope system to which the adaptor 200 is attached.

In accordance with embodiments of the present invention, variations in uterus size and shape, and location of the ostia to the fallopian tubes may be accommodated by choosing to attach an adaptor with a particular bend 218 and straight portion 222, if present, to the working channel of a hysteroscope system. Referring to FIGS. 5A-5B and in accordance with some embodiments of the invention, the angle of deflection the adaptor creates can be controlled. For example, the angle of deflection may be from 20 degrees to 90 degrees. As illustrated in FIG. 5A, the bend 218 deflects the central axis of the adaptor 20 degrees at the distal end 214. As illustrated in FIG. 5B, the bend 218 deflects the central axis of the adaptor 90 degrees at the distal end 214. Referring to FIGS. 6A-6B and in accordance with some embodiments of the invention, the bend 218 may have a controlled radius of curvature. For example, the radius of curvature may be from 0.020 inches (as illustrated in FIG. 6A) to 2 inches (as illustrated in FIG. 6B). Referring now to FIGS. 7A-7B and in accordance with some embodiments of the invention, the length of straight section 222, if present, can be tuned for a particular application or anatomy. As illustrated, straight section 222 in FIG. 7B is comparatively longer than straight section 222 in FIG. 7A. In an embodiment, straight section 222 may have a length of 0 cm to 20 cm.

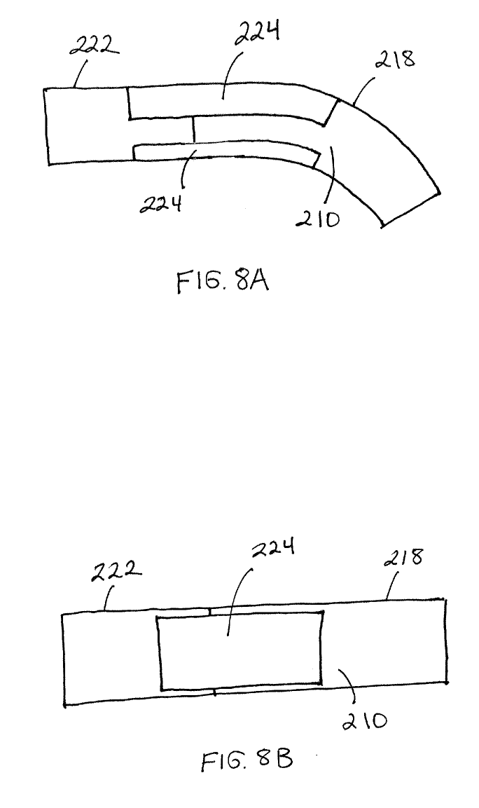

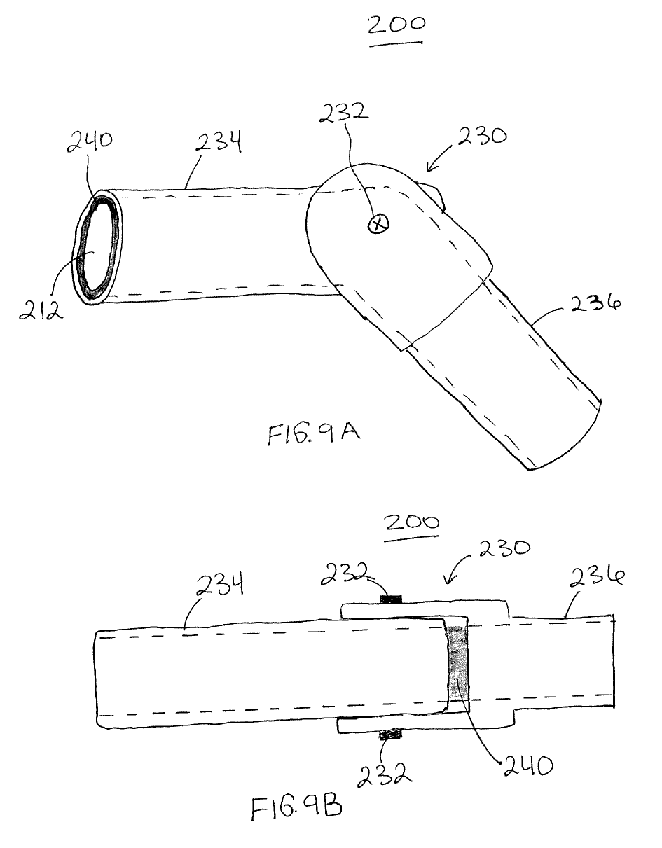

The elongate body 210 of adaptor 200 may be formed of a variety of different materials. In accordance with some embodiments, the elongate body 210 may include a transparent material. Utilizing the glass optics 112 of the hysteroscope, the transparent material may allow the user to visually see a catheter or other device being advanced from the working channel 114 of the hysteroscope and through the central lumen 212 of the adaptor 200. It is not required for the elongate body 210 to include a transparent material, and the elongate body may be formed of an opaque material in accordance with other embodiments. The elongate body 210 may also be formed of a combination of transparent and opaque materials. In one embodiment, straight section 222 may be formed of a transparent material, and bend 218 is formed of an opaque material. In another embodiment illustrated in FIGS. 8A-8B, the elongate body 210 may include a window 224 of transparent material through which the glass optics 112 of the hysteroscope system can view through. A window 224 may be located at any position along elongate body 210 in order to aid in visualization and provide a visualization path for the glass optics 112. Windows 224 may also be located on both sides of elongate body 210 in order to provide a visualization path through the elongate body 210 as illustrated in FIG. 8A.

The elongate body 210 may be formed of both rigid and flexible materials. For example, a rigid transparent material polymer such as polycarbonate, or a rigid transparent glass such as a tempered soda-lime glass, also known as pyrex® may be used. The elongate body 210 may also be formed of a rigid opaque material such as stainless steel. In an embodiment, the elongate body 210 is formed of a flexible material. A broad range of flexibilities are possible. In an embodiment, the elongate body 210 is formed of a polymer which is flexible to allow for easy cannulation of the cervical canal and cause less discomfort to a patient when advanced through the cervical canal, yet retains the intended shape and bend when advanced into a distended uterus. In an embodiment, the elongate body 210 is formed of a thin walled steel which can be manually deformed to achieve a desired bend by the operator. In another embodiment, the elongate body 210 may be formed of a shape memory polymer. In such an embodiment, the elongate body 210 may be inserted through the cervical canal and into the uterine cavity in a straight configuration (e.g. without a bend 218) thereby causing a minimum amount of discomfort to the patient during initial advancement. Then upon being warmed by a distention fluid (e.g. at body temperature) within the uterine cavity the elongate body 210 reconfigures to its memory shape having a desired bend 218.

FIGS. 9A-9B are illustrations of an adaptor 200 including an adjustable joint 230 in accordance with an embodiment of the invention. As illustrated, the adjustable joint 230 may include one or more set screws 232 to tighten proximal and distal arms 234, 236 together at a desired bend angle, though other mechanical configurations are contemplated in accordance with embodiments of the invention in order to tighten the adjustable joint 230. A tubular inner sleeve 240 with a smooth inner surface defining the central lumen 212 may be included within the proximal and distal arms 234, 236, which may also be tubular. In this manner, an operator may easily adjust the bend angle of the adaptor 200 at the point of use.

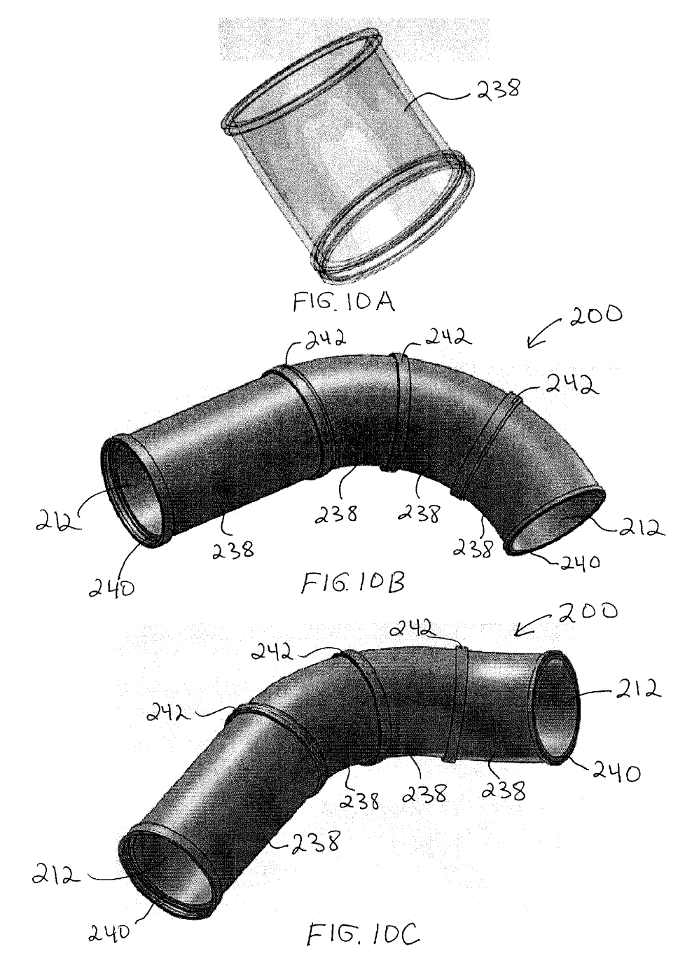

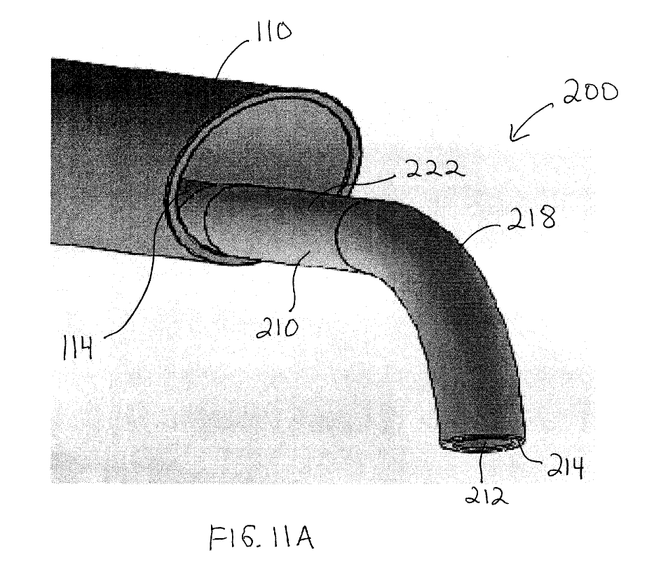

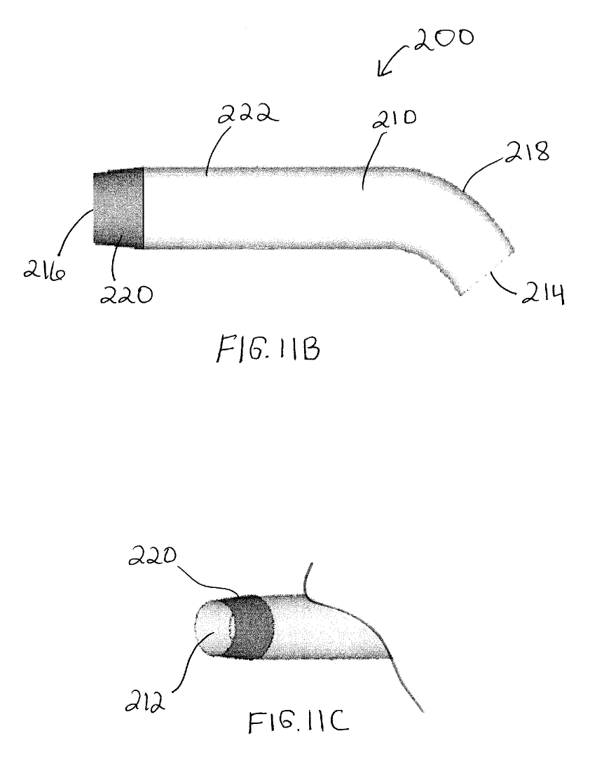

FIGS. 10A-10C are illustrations of adjustable cylinders 238 and adjustable adaptors 200 that may be formed from a plurality of connected adjustable cylinders 238, which an operator can adjust by twisting at their connections 242 in order to obtain a desired bend. The elongate body including a plurality of adjustable cylinders may include a tubular inner sleeve 240 with a smooth inner surface defining the central lumen 212. In this manner, an operator may easily adjust the bend angle of the adaptor 200 at the point of use.

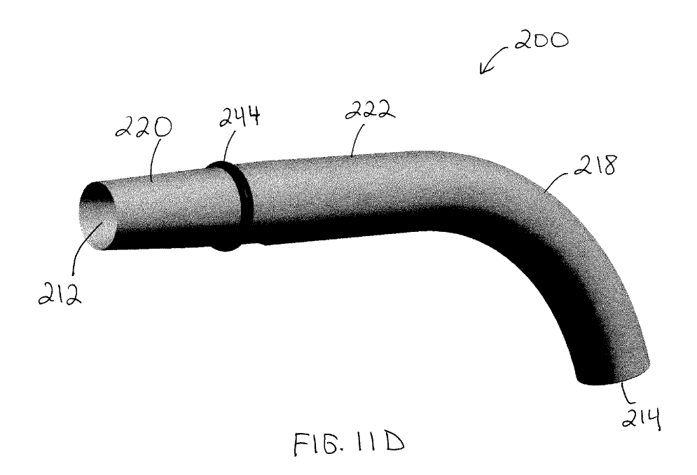

In accordance with embodiments of the invention, the adaptor 200 may be configured to be removably coupled to a working channel of a hysteroscope system. Referring now to FIGS. 11A-11D, the proximal end 216 of elongate body 210 may be configured to press fit into a working channel 114 of a hysteroscope system. Press fitting may both couple and secure the elongate body to the working channel 114. In an embodiment, proximal end 216 may include a taper 220 which is configured to couple with a working channel 114 of a hysteroscope system. In an embodiment, the proximal end 216 may fit on the inside of the working channel 114 such that the elongate body 210 extending from the working channel 114 has approximately the same outside diameter as the working channel 114. In an embodiment illustrated in FIG. 11D, an elastic material 244 such as silicone or urethane is located on the outside of the taper 220 such that the elastic material springs onto the inner surface of the working channel 114 when the adaptor 200 is press fit into the working channel 114. The elastic material may be an O-ring.

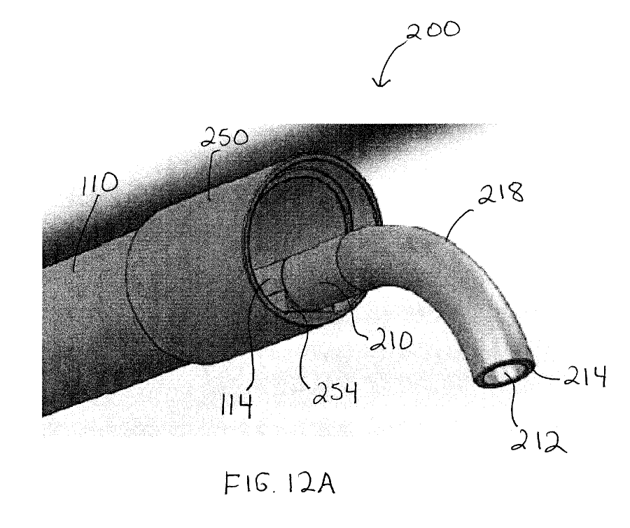

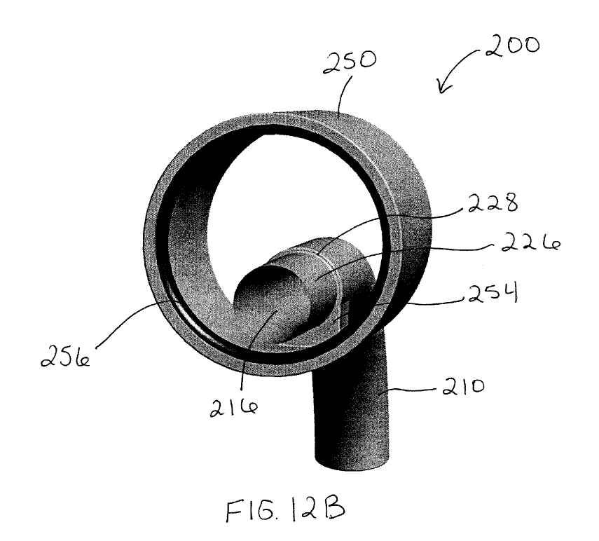

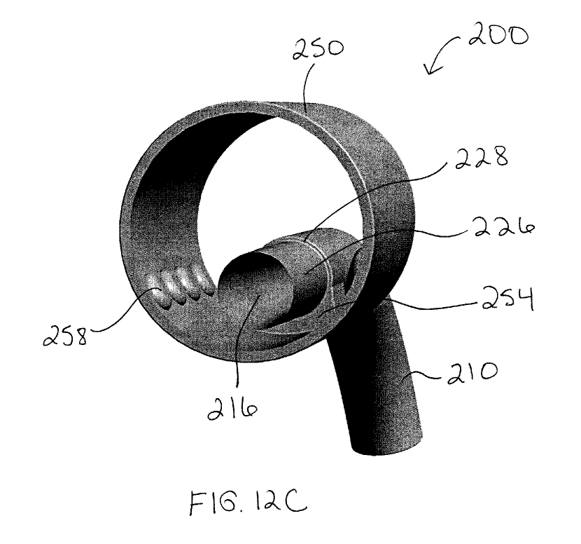

Referring now to FIGS. 12A-12C, the adaptor 200 may include a sleeve 250 which is configured to slip over a rigid elongated sheath 110 of a hysteroscope system to secure the adaptor 200 to the hysteroscope system. Sleeve 250 may be rigidly attached to the elongate body 210 with a bracket 254, for example. In an embodiment illustrated in FIG. 12B, sleeve 250 may be secured on the hystero scope system with an elastic material 256 on the inside of the sleeve 250 that springs onto the outside of rigid elongated sheath 110. For example, the elastic material may be in the shape of an O-ring, and may be formed of a suitable material such as silicone or urethane, amongst others. In an embodiment illustrated in FIG. 12C, sleeve 250 may include a plurality of protrusions 258 along a length of the sleeve 250 which are configured to align with and lock onto a corresponding plurality of side outflow openings 120 along a length of the rigid elongated sheath 110 to secure the adaptor 200 onto the hysteroscope system. As illustrated in FIGS. 12B-12C, a proximal portion 226 of elongate body 210 near the proximal end 216 may be configured to fit within a working channel 114 of a hysteroscope system. Proximal portion 226 may be tapered similarly as taper 220, or may alternatively have a constant diameter which is less than the diameter of the working channel 114. A backstop 228 representing a change in outer diameter of elongate body 210 may be provided to serve as an indicator for maximum insertion depth into the working channel 114 and may abut against a distal end surface of working channel 114.

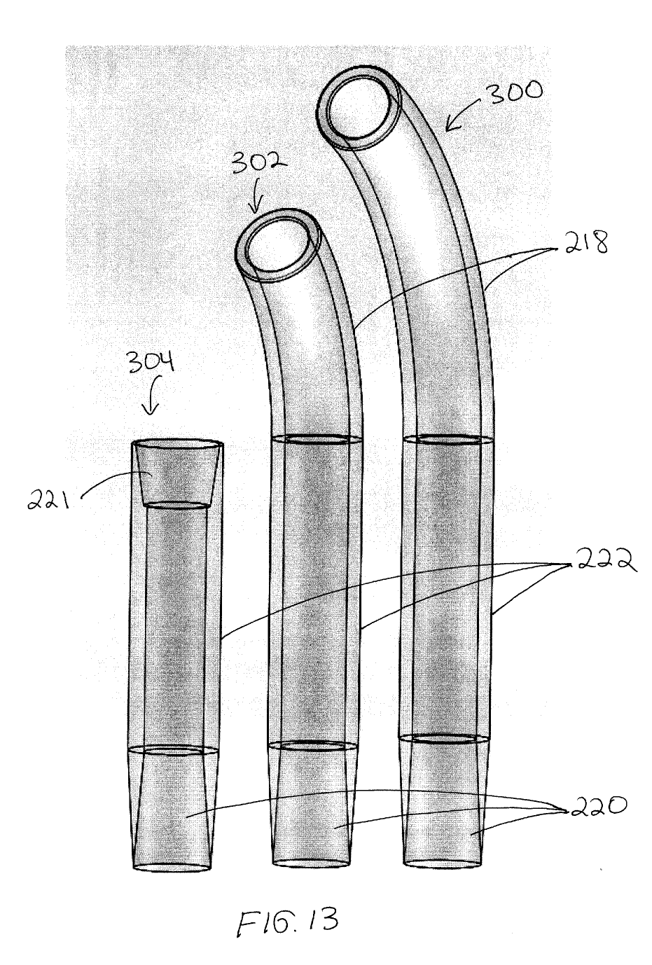

In accordance with embodiments of the present invention, a plurality of non-identical endoscope adaptors may be sold in a kit to obtain a suitable configuration for a particular patient anatomy. For example, the plurality of non-identical adaptors may include a plurality of different radii of curvature, a plurality of different angles of deflection, or a plurality of different straight portion lengths for the plurality of elongate bodies. A kit may include a plurality of non-identical adaptors having a number of combinations of different characteristics. FIG. 13 is an illustration of a press fit adaptor kit in accordance with embodiments of the invention. For example, a press fit adaptor kit may include a first adaptor 300 with a taper 220, a 0.4 inch straight portion 222, and a bend 218 with a 7 mm (0.276 inches) radius of curvature and a 75 degree angle of deflection; a second adaptor 302 with a tapper 220, a 0.4 inch straight portion 222, and a bend 218 with a 5 mm (0.197 inches) radius of curvature and a 60 degree angle of deflection; and an adaptor extension 304 with a taper 220 a 0.4 inch straight portion 222 and an outwardly tapered receiving portion 221 to receive a corresponding taper 220 of an adaptor 300, 302. It is to be appreciated that while specific ranges of radius of curvature, angle of deflection and straight portion or adaptor extension are described with regard to FIG. 13 that any combination of different characteristics can be provided in accordance with embodiments of the invention, and that those illustrated in FIG. 13 are to be regarded as illustrative rather than restrictive.

In an embodiment, an operator may select an adaptor based on assessment of the location of the ostia of the fallopian tubes, anatomy, and operator preference. If the ostia are lateral, then a shorter radius of curvature with a longer angle of deflection might be selected. If the cornua are deep, a longer adaptor might be used or if a polyp or myoma is blocking the ostia, a larger radius of curvature might be chosen.

Once the operator has selected an appropriate adaptor, the adaptor may be press fit into the working channel or slipped over the rigid elongated sheath of a hysteroscope system as described. The hysteroscope system may then be inserted into the uterine cavity, being careful while entering the cervical canal. The hysteroscope system with attached adaptor may need to be angled to maximize patient comport while tracking through the cervical canal. Once an ostium has been visualized, the operator can track a catheter system such as the Essure® system down the working channel of the hysteroscope system and visualize the catheter system being deflected by the adaptor. In accordance with embodiments of the present invention, the deflection the catheter system by the adaptor allows the operator to minimize the amount of hysteroscope system deflection while maximizing patient comfort. The catheter system may then be tracked into a fallopian tube to deposit an insert and removed. In accordance with embodiments of the invention, the operator may decide to switch adaptors to access the other ostia. If so, the hysteroscope system may be angled during removal to maximize patient comfort while the adaptor is removed through the cervical canal. The process may be repeated as necessary. The operator may alter the adaptor selection simply by pulling the adaptor off of the hysteroscope system and selecting a new adaptor.

In the foregoing specification, various embodiments of the invention have been described. It will, however, be evident that various modifications and changes may be made thereto without departing from the broader spirit and scope of the invention as set forth in the appended claims. The specification and drawings are, accordingly, to be regarded in an illustrative sense rather than a restrictive sense. Hence, the scope of the present invention is limited solely by the following claims.

What is claimed is:

1. An adaptor comprising:
an elongate body including a central lumen extending between a distal end and a proximal end of the elongate body, and a bend along a length of the elongate body between the distal and proximal ends;
wherein the proximal end is configured to be removably coupled with a working channel of an endoscope.

2. The adaptor of claim 1, wherein the bend deflects the central lumen between 0 and 90 degrees.

3. The adaptor of claim 2, wherein the bend has a radius of curvature between 0.020 inches to 2 inches.

4. The adaptor of claim 2, wherein the elongate body further comprises a straight portion.

5. The adaptor of claim 4, wherein the straight portion is between 0 cm and 20 cm.

6. The adaptor of claim 2, wherein the proximal end of the elongate body is configured to press fit into the working channel.

7. The adaptor of claim 6, wherein the proximal end is tapered.

8. The adaptor of claim 2, further comprising a sleeve attached to the elongate body, wherein the sleeve is configured to slip over an elongated sheath of the endoscope.

9. The adaptor of claim 8, wherein the sleeve comprises protrusions along a length of the sleeve which are configured to align with side outflow openings along a length of the elongated sheath.

10. The adaptor of claim 8, further comprising an elastic material on an inside of the sleeve that is configured to spring onto an outside of the elongated sheath.

11. The adaptor of claim 10, wherein the elastic material is an O-ring.

12. The adaptor of claim 10, wherein the elastic material comprises silicone or urethane.

13. The adaptor of claim 2, wherein the elongate body is transparent.

14. The adaptor of claim 2, wherein the elongate body comprises a transparent window.

15. The adaptor of claim 2, wherein the elongate body is flexible.

16. The adaptor of claim 2, wherein the elongate body is manually deformable to adjust the bend.

17. The adaptor of claim 2, wherein the elongate body is deformable by application of heat to adjust the bend.

18. The adaptor of claim 17, wherein the elongate body is formed of a shape memory material which deforms when inserted into a uterine cavity.

19. The adaptor of claim 17, wherein the shape memory material is transparent.

20. The adaptor of claim 2, wherein the elongate body is rigid.

21. The adaptor of claim 20, wherein the elongate body comprises a material selected from the group consisting of polycarbonate, glass and pyrex.

22. The adaptor of claim 2, wherein the elongate body comprises an adjustable joint.

23. The adaptor of claim 22, wherein the adjustable joint further comprises a set screw.

24. The adaptor of claim 2, wherein the elongate body comprises a plurality of connected adjustable cylinders.

25. The adaptor of claim 24, wherein the plurality of connected adjustable cylinders are adjustable by twisting.

26. A kit comprising:
a plurality of non-identical adaptors;
wherein each of the plurality of non-identical adaptors comprises:
an elongate body including a central lumen extending between a distal end and a proximal end of the elongate body, and a bend along a length of the elongate body between the distal and proximal ends;
wherein the proximal end is configured to be removably coupled with a working channel of an endoscope.

27. The kit of claim 26, wherein the plurality of non-identical adaptors comprise a plurality of different radii of curvature for the plurality of bends.

28. The kit of claim 26, wherein the plurality of non-identical adaptors comprise a plurality of different angles of deflection for the plurality of central lumens.

30. The kit of claim 26, further comprising a straight adaptor extension.
